(19) United States
(12) Patent Application Publication
(10) Pub. No.: US 2026/0355007 A1
(43) Pub. Date: Nov. 12, 2026

(54) HUMAN, OR ANIMAL, BODY IMMERSION BAG

(71) Applicant: NEREUS MEDICAL LTD, Exton, Exeter (GB)

(72) Inventor: Ross Hemingway, Exton, Exeter (GB)

(21) Appl. No.: 19/515,666

(22) PCT Filed: Sep. 11, 2024
(86) PCT No.: PCT/GB2024/052365

§ 371 Date: Mar. 2, 2026

(30) Foreign Application Priority Data
Sep. 15, 2023 (GB) 2314096.5

(51) Int. Cl. A61F 7/00

(52) U.S. Cl. CPC A61F 7/0053; A61F 2007/0059

(57) ABSTRACT

When a person or animal has a suspected or diagnosed heat illness, cold water immersion (CWI) is a known method of rapidly lowering the core body temperature. There is provided a human, or animal, body immersion device, wherein the length of the device is adjustable. The device comprises a bag a roll zone a stiffening element, and a fastening means comprising a first fastening component secured to the bag within the roll zone and a second fastening component secured to the bag outside the roll zone. In use, the stiffening element is rollable within the roll zone to adjust the length of the immersion bag, and the first fastening component is fastenable to the second fastening component to retain the human, or animal, body immersion device at its adjusted length.

### CROSS-REFERENCE TO RELATED APPLICATIONS

This application is a U.S. national phase of International Appl. No. PCT/GB2024/052365 filed Sep. 11, 2024, which claims priority to Great Britian Patent Appl. No. 2314096.5 filed Sep. 15, 2023, the contents of these applications are incorporated herein by reference in their entireties.

### TECHNICAL FIELD

The present invention relates generally to a human, or animal, body immersion bag, and a method of submerging a human body or an animal body within an immersion bag, and finds particular, although not exclusive, utility in adjusting the length of a human, or animal, body immersion bag.

### BACKGROUND

When a person or animal has a suspected or diagnosed heat illness such as heat exhaustion or heat stroke, cold water immersion (CWI) is a known method of rapidly lowering the core body temperature. CWI may also be carried out as a means of muscle recovery after exercising. Similarly, a person or animal may also be immersed in warm water to raise their core body temperature, or passively warmed with insulating material or warming devices.

It is known to immerse people or animals suffering from a heat illness in a rigid container, such as a bath tub, filled with water. However, rigid containers require a large amount of water to sufficiently immerse a person or relatively large animal. Furthermore, when filled with water, rigid containers may be heavy and cumbersome to move, such that a person or animal may not be easily transported within the rigid container, hindering the ability to take the person to a hospital, or the animal to a veterinarian. It may also be difficult to initially lift a person or relatively large animal into a rigid container, as they must be lifted over a rigid side, and then lowered into the container. Moreover, rigid containers may also take up a large amount of space to store.

As an alternative to rigid containers, immersion bags formed of a flexible material are known.

However, current immersion bags may still require a relatively large amount of water to sufficiently immerse a person or relatively large animal, and are often difficult to empty. Therefore, it is desirable to have an improved human or animal body immersion bag.

### SUMMARY

In a first aspect, the present invention provides a human, or animal, body immersion device for enabling a human body or animal body to be substantially submerged within a fluid, the human, or animal, body immersion device comprising: a bag arranged to receive a human body or an animal body, and fluid, in use, the bag comprising: a head end opposite to a foot end; a face aperture located adjacent to the head end, the face aperture arranged to receive at least part of the face of a human body or animal body, in use; an access port configured to extend from the face aperture to an access port terminus, wherein the access port is arranged to enable ingress or egress of a human body or an animal body into the bag; wherein, the immersion device is configured to be adjustable in length, and, to retain the adjusted length; the immersion device further comprising: a roll zone located between the terminus and the foot end of the bag, the roll zone comprised of a first material; a stiffening element arranged in the vicinity of the foot end of the bag, the stiffening element comprised of a second material having a greater stiffness than the first material, the stiffening element arranged to extend substantially perpendicular to the longitudinal axis of the bag; and a fastening means comprising a first fastening component secured to the bag within the roll zone, and a second fastening component secured to the bag outside the roll zone, wherein the first fastening component is releasably securable to the second fastening element; such that, in use, the stiffening element is rollable within the roll zone to adjust the length of the immersion device, and the first fastening component is fastenable to the second fastening component to thereby retain the immersion device at its adjusted length.

In this way, the effective volume of the human, or animal, body immersion device may be reduced by shortening its length by sealing the space substantially beyond a person's feet or an animal's back feet, in use, thereby preventing fluid entering the space. This may save water, and reduce the time in which the person or animal may be sufficiently immersed. This may be the case where the bag is long enough to fit an adult but only a child size is required. Alternatively, this may be the case where the bag is long enough to fit a large animal, such as a large dog, but only a size sufficient for a small dog is required. Furthermore, in use with the stiffening element rolled within the roll zone, the fastening means may prevent the immersion device unrolling to its original length as a result of the fluid within the bag providing pressure against the roll.

An animal may be any land mammal, for example, a dog, a horse, a donkey, or a cow. In use, the animal may lie on its side, or prone, within the bag.

The bag may be comprised of a flexible and water resistant material. For example, the bag may be comprised of a plastics material such as PVC. The bag may comprise a bottom panel and a top panel, wherein, in use, a human body or an animal body may be arranged to lie on top of the bottom panel, and under the top panel. The edges of the bottom panel and top panel may be directly connected. The panels may be integral with one another. Alternatively, side panels may extend between the edges of the bottom panel and edges of the top panel. The bag may comprise an internal volume between the bottom panel and the top panel. The bag may generally be oblong in shape.

A human body or an animal body being substantially submerged within a fluid contained within the bag may comprise the human body or animal body being fully submerged within the fluid, with the exception of their face. However, the human body or animal body being submerged at least up to their coronal (frontal) plane may be considered to be substantially submerged.

The fluid may comprise, for example, water, gel, gas, or foam.

The longitudinal axis of the bag may be the axis that extends parallel to the coronal (frontal) plane of the human body and perpendicular to the transverse plane of the human body, in use with a human body in the bag. In this way, the longitudinal axis may extend between the head end and foot end of the bag.

The access port may comprise an opening capable of receiving a human body or an animal body. The access port terminus may define a limit of the opening. The access port may be arranged within the front panel of the immersion bag. The access port may comprise an opening substantially parallel to the longitudinal axis of the immersion bag.

The access port may be releasably sealable. For example, the access port may comprise a zip fastener, hook and loop fasteners, buttons, toggles, poppers, or magnets.

The human, or animal, body immersion device being adjustable in length may comprise the device being configured to be maintained in a plurality of lengths. This may comprise a maximum length in which the roll zone is fully unrolled or unfolded, and a minimum length in which the roll zone is completely rolled or folded. The length of the immersion bag may be configured to be any length between the maximum length and the minimum length.

The human, or animal, body immersion device may have an unrolled or unfolded length between 1 m and 3 m. The immersion device predominantly for use with a human body may have an unrolled or unfolded length between 2 m and 3 m. The immersion device predominantly for use with an animal body may have an unrolled or unfolded length between 1 m and 2 m. The immersion device predominantly for use with an animal body may be approximately 0.85 m shorter than the length of the immersion device predominantly for use with a human body.

The stiffening element may be comprised of the same material as the bag, but the stiffening element may have a greater thickness. Additionally or alternatively, the stiffening element may be comprised of a different material to the bag. For example, the stiffening element may be comprised of metal, plastics, composites, or wood.

The stiffening element being arranged in the vicinity of the foot end may include the stiffening element being arranged on the extremity of the foot end. Alternatively, the stiffening element may be arranged spaced from the extremity of the foot end towards the head end. For example, there may be a section of immersion bag material between the extremity of the foot end and the stiffening element. The stiffening element may span the entire width of the immersion bag, or only a portion of the width of the immersion bag.

The first fastening means may comprise, for example, straps or webbing connected to a buckle, hook and loop fastener, buttons, poppers, or magnets. In the case of a buckle, for example, the first fastening component may comprise a male end, and the second fastening component may comprise a female end into which the male end may be securable. The straps or webbing may be impregnated with antibacterial and/or anti-fungal treatment.

In use, the stiffening element rolled within the material of the roll zone may act as a seal to substantially prevent fluid entering the rolled section of the bag, due to the force of the stiffening element pressing against each roll or fold. The first fastening means may then maintain the seal in its closed position, such that the pressure of the fluid in the bag may not force the rolled section to unroll or unfold and consequently lower the fluid level, thus requiring more fluid to maintain the level.

The stiffening element may comprise a sealable aperture, or the sealable aperture may be arranged adjacent to the stiffening element, wherein the sealable aperture is arranged to either prevent the release of fluid from the bag, or controllable release the fluid from the bag, in use. The sealable aperture may be the stiffening element.

By having the sealable aperture arranged adjacent to the foot end, fully opening the sealable aperture may enable a rapid, yet controlled, expulsion of substantially all the fluid from the bag. In this way, a user does not need to attempt to empty the fluid from the bag via the access port, which may result in unwanted spillage of the fluid, or prevent all the fluid being easily removed.

When used in medical scenarios, it is possible that the fluid within the bag may be contaminated by bodily fluids such as feces, urine, vomitus and blood, which may pose a risk of infection, or contaminate the surrounding environment upon release. By providing the sealable aperture to control the release of the fluid into a container or drain, for example, the risk of infection and/or contamination, may be reduced.

The sealable aperture may comprise a zip fastener. In this way, the stiffening element may be a zip fastener. Alternatively, the zip fastening may be located adjacent to the stiffening element. In use, the release of fluid from the bag may be controllable by controlling the proportion of the zip fastener that is opened. It will be appreciated that other sealable means are contemplated, for example hook and loop fasteners, buttons, toggles, poppers, or magnets. The sealable means may be waterproof.

The first fastening component may be connected to a first side of the bag, and the second fastening component may be connected to a second side of the bag, the second side being opposite to the first side, such that, in use with the first fastening component secured to the second fastening component, the first fastening means is extended diagonally across the roll zone. The first fastening means may also extend across a portion of the human body immersion device not within the roll zone.

In this way, the first fastening component may extend across the centre of the rolled section, in use with the stiffening element rolled within the roll zone, thereby increasing the resistance to unrolling or unfolding caused by the pressure of the fluid within the immersion bag. The first fastening means may maintain the human, or animal, body immersion device in its rolled up or folded up state.

The first and second sides of the bag may be adjacent to the edges of the bottom and/or top panels.

The human, or animal, body immersion device may further comprise at least one stiffening panel arranged to substantially maintain the structural integrity of the bag, such that in use the fluid may maintain a desired minimum depth with the human, or animal, body immersion device arranged such that its longitudinal axis is in a substantially horizontal plane. The at least one stiffening panel may be arranged on a side of the bag. The stiffening panel may be arranged on the top panel of the bag. The stiffening panel may be arranged adjacent to an edge of the top panel of the bag. The stiffening panel may extend in a direction substantially parallel to the longitudinal axis of the immersion bag.

In use, with a stiffening panel arranged on a side of the bag, the width of the bag may be prevented from increasing with a corresponding reduction in height, and thus remain substantially constant, as fluid is introduced inside the bag. As such, less fluid may be required to provide a minimum depth of fluid within the bag to sufficiently immerse a human body or an animal body, than if the width of the bag were to increase with the introduction of fluid.

In this way, the stiffening panel may resist the force applied by the fluid within the bag causing the immersion bag to bulge or expand, thus requiring less fluid to sufficiently immerse a human body or animal body, in use.

The stiffening panel may be comprised of the same material as the bag, but the stiffening panel may have a greater thickness. Additionally or alternatively, the stiffening element may be comprised of a different material to the bag. For example, the stiffening element may be comprised of metal, plastics, composites, or wood.

The human, or animal, body immersion device may comprise a plurality of stiffening panels. For example, a stiffening panel may be arranged on each side of the bag.

The human, or animal, body immersion device may further comprise a means for reducing the effective volume of the bag. For instance, a plurality of length adjustable straps extending from one side of the bag to the other side of the bag, such that, in use, reducing the length of the length adjustable straps may act to constrict the bag around a human body or animal body and thus reduce its internal volume.

The length adjustable straps may be arranged parallel to each other, and perpendicular to the longitudinal axis of the immersion bag. The length adjustable straps may be arranged spaced apart along the length of the immersion bag.

The length adjustable straps may comprise buckles or ratchets to adjust the length. Alternatively, the adjustable straps may comprise a drawstring mechanism, elastic sections, or other resilient sections.

The length adjustable straps may extend circumferentially around the bag, such that, in use, the straps may provide an even constriction around the whole circumference of the bag. In use, with the length adjustable straps and the stiffening panels present, the stiffening panels may prevent the width of the bag between the length adjustable straps from increasing, such that the width of the bag may remain constant as fluid is introduced into the bag. In this way, the bag may not bulge between length adjustable straps.

The human, or animal, body immersion device may further comprise handles for carrying the bag with a human body or an animal body, and fluid, within. The handles may extend outwardly from the sides of the bag. The handles may comprise for example, loops of flexible material, or rigid handles.

A support portion may be arranged to extend from a handle located on one side of the bag to a handle located on the other side of the bag, such that, in use, the weight of the human body or animal body may be at least partially supported by the support portion.

The support portion may comprise a strap or webbing formed of a flexible material. Alternatively or additionally, the support portion may comprise a rigid material, such as plastics, metal, or wood. The straps or webbing may be impregnated with antibacterial and anti-fungal treatment.

In use, the support portion may be located beneath a human body or an animal body within the bag, such that when the bag is lifted via the handles, the human body or animal body is supported by the support portion rather than the bag. In this way, the chances of the bag tearing or stretching may be reduced.

The support portion may be fixedly secured to the bag to prevent the bag moving relative to the support portion, in use. In this way, the weight of the bag, including the fluid and human body or animal body, may be evenly distributed between handles and support portions. As such, the chance of the weight shifting and the bag being dropped as a result is reduced.

The bag may comprise a plurality of support portions.

In a second aspect, the invention provides a human body immersion device for enabling a human body to be substantially submerged within a fluid, the human body immersion device comprising: a bag arranged to receive a human body and fluid in use, the bag comprising: a head end opposite to a foot end; a face aperture located adjacent to the head end, the face aperture arranged to receive at least part of the face of a human body, in use; an access port configured to extend from the face aperture to an access port terminus, wherein the access port is arranged to enable ingress or egress of a human body into the bag; wherein, the immersion device is configured to be adjustable in length, and, to retain the adjusted length; the immersion device further comprising: a roll zone located between the terminus and the foot end of the bag, the roll zone comprised of a first material; a stiffening element arranged in the vicinity of the foot end of the bag, the stiffening element comprised of a second material having a greater stiffness than the first material, the stiffening element arranged to extend substantially perpendicular to the longitudinal axis of the bag; and a fastening means comprising a first fastening component secured to the bag within the roll zone, and a second fastening component secured to the bag outside the roll zone, wherein the first fastening component is releasably securable to the second fastening element; such that, in use, the stiffening element is rollable within the roll zone to adjust the length of the immersion device, and the first fastening component is fastenable to the second fastening component to thereby retain the immersion device at its adjusted length.

It will be appreciated that any of the optional features described above in relation to the first aspect may be included in the second aspect, such that they apply to an immersion device configured purely for a human body.

In a third aspect, the invention provides an animal body immersion device for enabling an animal body to be substantially submerged within a fluid, the animal body immersion device comprising: a bag arranged to receive an animal body and fluid in use, the bag comprising: a head end opposite to a foot end; a face aperture located adjacent to the head end, the face aperture arranged to receive at least part of the face of an animal body, in use; an access port configured to extend from the face aperture to an access port terminus, wherein the access port is arranged to enable ingress or egress of an animal body into the bag; wherein, the immersion device is configured to be adjustable in length, and, to retain the adjusted length; the immersion device further comprising: a roll zone located between the terminus and the foot end of the bag, the roll zone comprised of a first material; a stiffening element arranged in the vicinity of the foot end of the bag, the stiffening element comprised of a second material having a greater stiffness than the first material, the stiffening element arranged to extend substantially perpendicular to the longitudinal axis of the bag; and a fastening means comprising a first fastening component secured to the bag within the roll zone, and a second fastening component secured to the bag outside the roll zone, wherein the first fastening component is releasably securable to the second fastening element; such that, in use, the stiffening element is rollable within the roll zone to adjust the length of the immersion device, and the first fastening component is fastenable to the second fastening component to thereby retain the immersion device at its adjusted length.

It will be appreciated that any of the optional features described above in relation to the first aspect may be included in the third aspect, such that they apply to an immersion device configured purely for an animal body.

In a fourth aspect, the invention provides a method for immersing a human body or an animal body within a fluid contained within a human, or animal, body immersion device, the method comprising the steps of: providing the human, or animal, body immersion device of the first aspect; manipulating a human body or an animal body into the bag through the access port; rolling the stiffening element within the roll zone until the human, or animal, body immersion device is of a desired length; fastening the first fastening component to the second fastening component; and at least partially filling the bag with a fluid.

### BRIEF DESCRIPTION OF THE DRAWINGS

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

FIG. 1 is a plan view of the top of a human, or animal, body immersion device in an unrolled state.

FIG. 2 is a plan view of the top of a human, or animal, body immersion device maintained at a reduced length.

FIG. 3 is a plan view of the bottom of a human, or animal, body immersion device.

### DETAILED DESCRIPTION

The present invention will be described with respect to certain drawings but the invention is not limited thereto but only by the claims. The drawings described are only schematic and are non-limiting. Each drawing may not include all of the features of the invention and therefore should not necessarily be considered to be an embodiment of the invention. In the drawings, the size of some of the elements may be exaggerated and not drawn to scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that operation is capable in other sequences than described or illustrated herein. Likewise, method steps described or claimed in a particular sequence may be understood to operate in a different sequence.

Moreover, the terms top, bottom, over, under and the like in the description and the claims are used for descriptive purposes and not necessarily for describing relative positions. It is to be understood that the terms so used are interchangeable under appropriate circumstances and that operation is capable in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “an embodiment” or “an aspect” means that a particular feature, structure or characteristic described in connection with the embodiment or aspect is included in at least one embodiment or aspect of the present invention. Thus, appearances of the phrases “in one embodiment”, “in an embodiment”, or “in an aspect” in various places throughout this specification are not necessarily all referring to the same embodiment or aspect, but may refer to different embodiments or aspects. Furthermore, the particular features, structures or characteristics of any one embodiment or aspect of the invention may be combined in any suitable manner with any other particular feature, structure or characteristic of another embodiment or aspect of the invention, as would be apparent to one of ordinary skill in the art from this disclosure, in one or more embodiments or aspects.

Similarly, it should be appreciated that in the description various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Moreover, the description of any individual drawing or aspect should not necessarily be considered to be an embodiment of the invention. Rather, as the following claims reflect, inventive aspects lie in fewer than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some features included in other embodiments, combinations of features of different embodiments are meant to be within the scope of the invention, and form yet further embodiments, as will be understood by those skilled in the art. For example, in the following claims, any of the claimed embodiments can be used in any combination.

In the description provided herein, numerous specific details are set forth. However, it is understood that embodiments of the invention may be practised without these specific details. In other instances, well-known methods, structures and techniques have not been shown in detail in order not to obscure an understanding of this description.

In the discussion of the invention, unless stated to the contrary, the disclosure of alternative values for the upper or lower limit of the permitted range of a parameter, coupled with an indication that one of said values is more highly preferred than the other, is to be construed as an implied statement that each intermediate value of said parameter, lying between the more preferred and the less preferred of said alternatives, is itself preferred to said less preferred value and also to each value lying between said less preferred value and said intermediate value.

The use of the term “at least one” may mean only one in certain circumstances. The use of the term “any” may mean “all” and/or “each” in certain circumstances.

The principles of the invention will now be described by a detailed description of at least one drawing relating to exemplary features. It is clear that other arrangements can be configured according to the knowledge of persons skilled in the art without departing from the underlying concept or technical teaching, the invention being limited only by the terms of the appended claims.

The following description of the Figures refers to the use of the human, or animal, immersion device with a human body. However, it will be appreciated that an animal body is also contemplated.

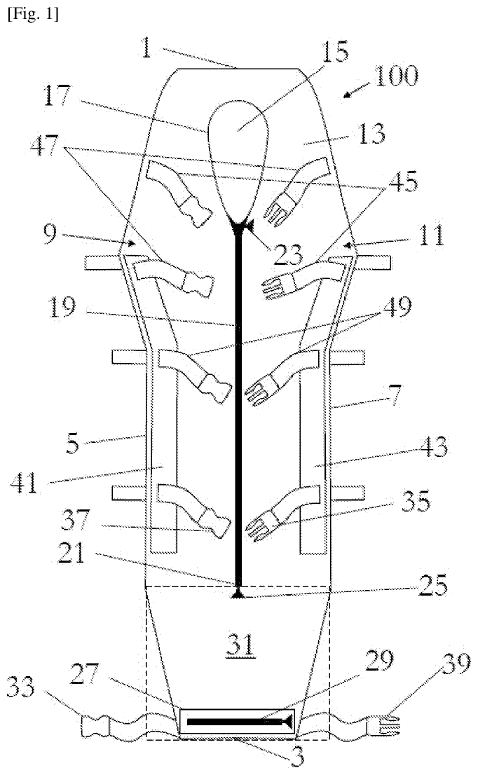

FIG. 1 shows a plan view of the top of a human, or animal, body immersion device in an unrolled state. The human, or animal, body immersion device comprises a bag 100. The bag 100 has a head end 1 opposite to a foot end 3. A first side 5 and a second side 7 extend between the head end 1 and foot end 3. The widths of the head end 1 and foot end 3 are less than the lengths of the first and second sides 5, 7. In this way, the bag 100 has a substantially elongate shape, wherein the length of the immersion bag is substantially greater than its width. The longitudinal axis may be the axis extending from the centre point of the head end 1 to the centre point of the foot end 3.

The width of the bag 100 is shown as being not constant along the length of the bag. The width of the bag 100 is greatest in the region of the bag 100 that in use may accommodate the shoulders of a person, thereby forming first and second shoulder regions 9, 11 located between the head end 1 and the centre of the bag 100. The width of the bag 100 is smallest in the region of the bag 100 that in use may accommodate the feet of a person. It is also contemplated that the width of the bag 100 may be uniform along its length.

The bag 100 comprises a top panel 13 formed as a single panel defining the shape of the bag 100 in plan view. The bag further comprises a bottom panel 15 formed as a single panel. The top and bottom panels 13, 15 are aligned such that their edges may be connected to each other to create an internal volume between the panels 13, 15.

A face aperture 17 is located within the top panel 13 adjacent to the head end 1, and is substantially ovular in shape. However, other shapes are also contemplated, such as a circular or square shape. The face aperture 17 is located in a region of the bag 100 that is arranged, in use, to accommodate the face of a person. In this way, the dimensions of the face aperture 17 may be sufficient to receive at least a portion of the face of a person within the bag 100.

Also shown is an access port 19 within the top panel 13 of the bag 100. The access port 19 extends from the face aperture 17 to an access port terminus 21. However, it is also contemplated that access port 19 may not be connected to the face aperture 17. The access port terminus 21 is located in the region of the bag 100 which in use may accommodate the feet of a person. The access port terminus 21 is shown to be located spaced away from the foot end 3. For example, the access port terminus 21 may be located 300 mm to 600 mm from the foot end 3.

The access port 19 may be merely an elongate opening. However, the access port 19 may be releasably sealable to prevent, in use, water escaping from the bag 100. One possible means for releasably sealing the access port 19 is a zip fastener. FIG. 1 shows a first toggle 23 for a zip fastener located at an end of the access port 19 adjacent to the face aperture 17. In use, the first toggle 23 enables the access port 19 to be opened. A second toggle 25 for a zip fastener is shown located adjacent to the access port terminus 21. In use, the second toggle 25 may be used to unseal a portion of the access port 19 adjacent to the access port terminus 21, whilst maintaining the rest of the access port 19 sealed. In this way, the legs and/or feet of the human body may be positioned outside the bag 100 to relieve cramp or other pains resultant from cold water immersion.

It will be appreciated that other means for releasably sealing the access port 19 are also contemplated. For example, hook and loop fasteners, buttons, toggles, poppers, or magnets may be utilised. It will be further appreciated that the other releasable sealing means may also enable a section of the access port 19 adjacent to access port terminus 21 to be unsealed, whilst the rest of the access port remains sealed.

Whilst the face aperture 17 and the access port 19 are described as two separate elements, it is also contemplated that the face aperture 17 may alternatively be an unsealed section of the access port 19.

The bag 100 further comprises a stiffening element 27. The stiffening element 27 is located adjacent to the foot end 3, and extends perpendicular to the bag's longitudinal axis. The stiffening element 27 extends from the first side 5 to the second side 7. However, it is also contemplated that the stiffening element 27 may only extend across a portion of the width of the bag.

The stiffening element 27 may be comprised of a material having a greater stiffness than the material of the bag 100. For example, the stiffening element 27 may be comprised of the same material as the bag 100, but the stiffening element 27 may have a greater thickness. Additionally or alternatively, the stiffening element may be comprised of metal, plastics, composites, or wood.

The stiffening element 27 in FIG. 1 is shown as comprising a zip fastener 29. The zip fastener extends perpendicularly to the longitudinal axis, and parallel to the extension of the stiffening element 27. In this way, with the bag 100 unrolled, a user may release fluid from the bag 100 in a controlled manner. For example, opening the zip fastener 29 by a relatively small amount may allow a small flow of fluid to be released. By comparison, fully opening the zip fastener 29 may allow a large flow of fluid to be released.

A roll zone 31 is provided between the access port terminus 21 and the foot end 3, and is indicated by the dashed box. In FIG. 1, the bag 100 is shown to be unrolled, and therefore at its maximum length.

First and second fastening means are shown. The first fastening means is formed of a first buckle female part 33 located on a strap extending from the first side 5 within the roll zone 31 and adjacent to the stiffening means 27, and first buckle male part 35 located on a strap extending from the second side 7 at a point outside the roll zone 31. The second fastening means is formed of a second buckle female part 37 located on a strap extending from the second side 7 at a point outside the roll zone 31, and second buckle male part 39 located on a strap extending from the second side 7 within the roll zone 31 and adjacent to the stiffening means 27. The two fastening means are shown to be unfastened, such that the first buckle male part 35 is spaced away from, and not secured within the first buckle female part 33. Similarly, the second buckle male part 39 is spaced away from, and not secured within the second buckle female part 37.

Also shown are stiffening panels 41, 43. The stiffening panels are located on the top panel 13 and adjacent to the first and second sides 5, 7, of the bag 100 respectively. The stiffening panels 41, 43 extend substantially parallel to the longitudinal axis of the bag 100. The stiffening panels 41, 43 may be comprised of a material having a greater stiffness than the material of the bag 100. For example, the stiffening panels 41, 43 may be comprised of the same material as the bag 100, but the stiffening element 27 may have a greater thickness. Additionally or alternatively, the stiffening element may be comprised of metal, plastics, composites, or wood.

With the bag 100 empty of fluid and a person, the stiffening panels 41, 43 may lie parallel to the surface on which the bag 100 may be supported. In use, when the bag 100 is being filled with fluid, the stiffening panels 41, 43 may rotate away from the surface, such that they may be perpendicular relative to the surface once filled.

FIG. 1 also shows three pairs of length adjustable straps 45, 47, 49. The first strap of each of the three pairs of length adjustable straps 45, 47, 49 is attached to the immersion bag adjacent to the first side 5. The second strap of each of the three pairs of length adjustable straps 45, 47, 49 is attached to the immersion bag adjacent to the second side 7. The first strap of each of the three pairs of length adjustable straps 45, 47, 49 comprises a buckle female part. The second strap of each of the three pairs of length adjustable straps 45, 47, 49 comprises a buckle male part.

It will be appreciated that there may be more or less than three pairs of length adjustable straps. For example, there may be one, two, four, or five pairs of length adjustable straps, or more. It will be further appreciated that the length adjustable straps may comprise releasable securing means other than buckles. For example, hook and loop fasteners, buttons, toggles, or poppers may be used.

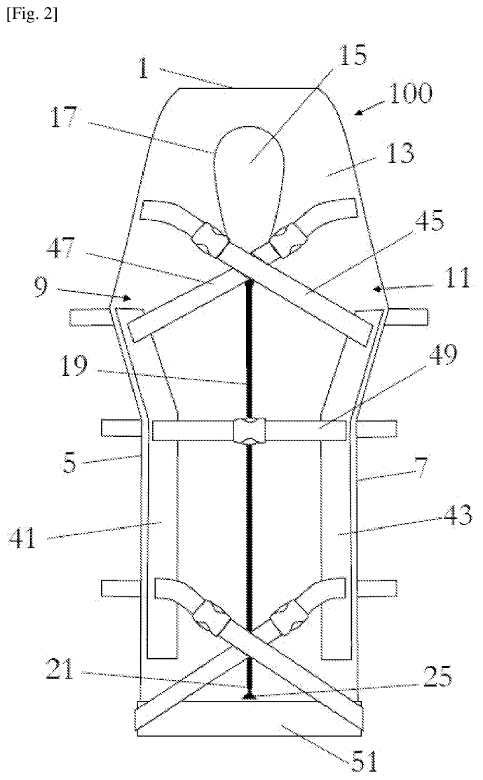

FIG. 2 shows a plan view of the top of a human, or animal, body immersion device maintained at a reduced length.

The stiffening element 27 shown in FIG. 1 is not visible in FIG. 2. This is because it has been rolled within the roll zone 31, resulting in a rolled section 51.

The first and second fastening means are shown to be fastened. In this way, the first buckle male part 35 is secured within the first buckle female part 33. As such, the straps of the first fastening means are shown to extend diagonally across the rolled section 51 from the first side 5 to the second side 7. Similarly, the second buckle male part 39 is secured within the second buckle female part 37. As such, the straps of the second fastening means are shown to extend diagonally across the rolled section from the second side 5 to the first side 5. In this way, the reduced length of the bag may be retained, thus maintaining the bag 100 in a reduced volume state. Accordingly, that the volume of fluid required to maintain the depth of the fluid at a level sufficient to immerse a person is reduced as compared to the bag in an un-shortened state. Furthermore, the weight of the bag 100 including fluid and a person may be reduced, such that the human body immersion device may be easier to carry.

The three pairs of length adjustable straps 45, 47, 49 are shown to be secured across the width of the bag 100. The pairs of length adjustable straps 45, 47 are shown to extend diagonally across the width of the bag 100. In use, the pairs of length adjustable straps 45, 47 may cross the chest of a person within the bag 100. In use, the pair of length adjustable straps 49 may extend substantially across the waist or midriff of a person within the bag 100.

The second toggle 25 is still visible in FIG. 2. In this way, a section of the access port 19 adjacent to the access port terminus 21 may still be openable such that the legs and/or feet of the person may be placed outside of the bag 100 to relieve cramp or other pains resultant from cold water immersion.

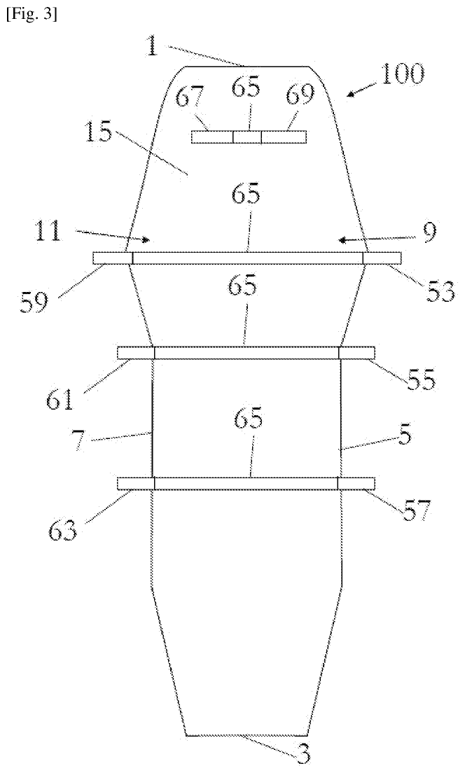

FIG. 3 shows a plan view of the bottom of the human, or animal, body immersion device. First handles 53, 55, 57 are shown extending away from the first side 5 of the bag 100. Second handles 59, 61, 63 are shown extending away from the second side 7. First handle 53 is opposite to second handle 59. First handle 55 is opposite to second handle 61. First handle 57 is opposite to second handle 63. The handles may be comprised of a flexible material, and for example may comprise looped straps or webbing. It will be appreciated that rigid handles are also contemplated.

A support portion 65 is shown extending between each of the opposing handles. Specifically, a support section 65 extends between first handle 53 and second handle 59, another support section 65 extends between first handle 55 and second handle 61, and a further support section 65 extends between first handle 57 and second handle 63.

The support portions 65 may be comprised of the same flexible material as the first handles 53, 55, 57 and second handles 59, 61, 63. In this way, the support portions and handles may comprise a singular, continuous strap extending across the width of the immersion bag. Alternatively, the support portions 65 may comprise a rigid material, such as plastics, metal, or wood. In this way, the first handles 53, 55, 57 and second handles 59, 61, 63 may be secured to the support portions 65. The support portions may be fixedly attached to the bag 100, such that they do not move relative to the bag 100, in use.

In use, support portions 65 may be located beneath a person within the bag 100, such that when the bag 100 is lifted via the handles, the person may be supported by the support portions rather than the bag 100. In this way, the chances of the bag 100 tearing or stretching may be reduced.

Additional head handles 67, 69 are also shown in FIG. 3 adjacent to the head end 1. A support portion 65 is shown to extend between the head handles 65, 67. In use, the head handles 67, 69 may be used predominantly to support the person's head when being carried in the bag 100.

In use, the bag 100 of FIG. 1 may be laid flat on the ground, or other relatively flat surface. The access port 19 may be opened via the first toggle 23, and a person may be placed within the bag 100. The access port 19 may then be at least partially sealed via the first toggle 23.

Once the person is within the immersion bag, the stiffening element 27 may be rolled within the roll zone 31 until the rolled section 51 is adjacent to the feet of the person. Once rolled, the rolled section 51 may be maintained in position by securing the first buckle male part 35 within the first buckle female part 33, and securing the second buckle male part 39 within the second buckle female part 37. In this way, the rolled section 51 of the bag 100 may be sealed, reducing the effective volume of the bag 100, and preventing water from entering the rolled section 51.

Next, the pairs of length adjustable straps 45, 47 may be secured across the chest of the person, and the pair of length adjustable straps 49 may be secured across the waist of the person. The length of the pairs of length adjustable straps 45, 47, 49 may then be reduced. In this way, the effective volume of the bag 100 may be further reduced by constricting the bag 100 around the person, thus requiring less fluid to maintained the required depth of the fluid.

Water, or any other fluid, may then be poured into the bag 100 through a partially opened section of the access port 19, thereby immersing the person.

Should the person require transportation to a hospital, or elsewhere, the bag 100 may be carried via the first and second handles 53, 55, 57, 59, 61, 63. In this way, the weight of the person and the water may be supported by the support portions 65.

If the person begins to suffer from cramping legs, the second toggle 25 may be used to open a section of the access port 19 adjacent to the person's feet, such that the person's feet may be lifted out of the immersion bag, to relieve the symptoms of cramp.

When the bag 100 is to be emptied, the first and second fastening means may be unbuckled, the rolled section 51 may be unrolled, and the zip fastener 29 may be opened to allow a small flow of water to be released, or fully opened to allow a large flow of water to be released.

In this way, a person may be placed within an immersion bag, and the effective volume of the immersion bag may be reduced by shortening the length of the bag, and retaining the shortened length, such that less fluid may be required to reach a fluid level within the bag sufficient to immerse the person.

It will be appreciated that an animal body, such as the body of a dog, may also utilise the human, or animal, body immersion device. When used for a dog, the dog may lie on its side, or prone, within the bag 100. In this way, the dog's face and/or head may extend at least partially through the face aperture 17, and the dog's hind legs may be arranged in the foot end 3 of the bag 100.

1-15. (canceled)

16. A human, or animal, body immersion device for enabling a human body or an animal body to be substantially submerged within a fluid, the human, or animal, body immersion device comprising:
a bag arranged to receive a human body or an animal body, and fluid, in use, the bag comprising:
a head end opposite to a foot end;
a face aperture located adjacent to the head end, the face aperture arranged to receive at least part of the face of a human body or an animal body, in use;
an access port configured to extend from the face aperture to an access port terminus, wherein the access port is arranged to enable ingress or egress of a human body or animal body into the bag;
wherein, the human, or animal, body immersion device is configured to be adjustable in length, and, to retain the adjusted length;
the human, or animal, body immersion device further comprising:
a roll zone located between the terminus and the foot end of the bag, the roll zone comprised of a first material;
a stiffening element arranged in the vicinity of the foot end of the bag, the stiffening element comprised of a second material having a greater stiffness than the first material, the stiffening element arranged to extend substantially perpendicular to the longitudinal axis of the bag; and
a fastener comprising a first fastening component secured to the bag within the roll zone, and a second fastening component secured to the bag outside the roll zone, wherein the first fastening component is releasably securable to the second fastening element;
such that, in use, the stiffening element is rollable within the roll zone to adjust the length of the human, or animal, body immersion device, and the first fastening component is fastenable to the second fastening component to thereby retain the immersion device at its adjusted length.

17. The human, or animal, body immersion device of claim 16, wherein the stiffening element comprises a sealable aperture arranged to either prevent the release of fluid from the bag, or controllably release the fluid from the bag, in use.

18. The human, or animal, body immersion device of claim 16, wherein a sealable aperture is arranged adjacent to the stiffening element, wherein the sealable aperture is arranged to either prevent the release of fluid from the bag, or controllable release the fluid from the bag, in use.

19. The human, or animal, body immersion device of claim 17, wherein the sealable aperture comprises a zip fastener.

20. The human, or animal, body immersion device of claim 16, wherein the first fastening component is connected to a first side of the bag, and the second fastening component is connected to a second side of the bag, the second side being opposite to the first side, such that, in use with the first fastening component secured to the second fastening component, the fastener is extended diagonally across the roll zone.

21. The human, or animal, body immersion device of claim 16, further comprising at least one stiffening panel arranged to substantially maintain the structural integrity of the bag, such that in use the fluid maintains a desired minimum depth with the bag arranged such that its longitudinal axis is in a substantially horizontal plane.

22. The human, or animal, body immersion device of claim 21, wherein the at least one stiffening panel is arranged on a side of the bag.

23. The human, or animal, body immersion device of claim 16, further comprising a means for reducing the effective volume of the bag.

24. The human, or animal, body immersion device of claim 23, wherein the means for reducing the effective volume of the bag comprises a plurality of length adjustable straps extending from one side of the bag to the other side of the bag, such that, in use, reducing the length of the length adjustable straps acts to constrict the bag around a human body or an animal body and thus reduce the internal volume of the bag.

25. The human, or animal, body immersion device of claim 24, wherein the length adjustable straps extend circumferentially around the bag, such that, in use, the straps provide an even constriction around the whole circumference of the bag.

26. The human, or animal, body immersion device of claim 16, further comprising handles for carrying the bag with a human body or an animal body, and fluid, within.

27. The human, or animal, body immersion device of claim 26, wherein a support portion is arranged to extend from a handle located on one side of the bag to a handle located on the other side of the bag, such that, in use, the weight of the human body or animal body is at least partially supported by the support portion.

28. The human, or animal, body immersion device of claim 27, wherein the support portion is fixedly secured to the bag to prevent the bag moving relative to the support portion, in use.

29. The human, or animal, body immersion device of claim 27, comprising a plurality of support portions.

30. A method for immersing a human body or an animal body within a fluid contained within a human, or animal, body immersion device, the method comprising the steps of:
providing the human, or animal, body immersion device of claim 16;
manipulating a human body or an animal body into the bag through the access port;
rolling the stiffening element within the roll zone until the human or animal body immersion device is of a desired length;
fastening the first fastening component to the second fastening component; and at least partially filling the bag with a fluid.
